(19) United States
(12) Patent Application Publication
(10) Pub. No.: US 2009/0264387 A1
(43) Pub. Date: Oct. 22, 2009

(54) ESTRA-1,3,5(10)-TRIENE DERIVATIVES FOR CONTRACEPTION

(75) Inventors: Tamara Pfaff, Dusseldorf (DE); Thomas Otten, Roetgen (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, AACHEN (DE)

(21) Appl. No.: 12/419,084

(22) Filed: Apr. 6, 2009

(30) Foreign Application Priority Data
Apr. 8, 2008 (EP) 08006956.0

(51) Int. Cl. A61K 31/566; A61K 31/56; A61P 15/18

(52) U.S. Cl. 514/170; 514/182

(57) ABSTRACT

The invention relates to pharmaceutical compositions and administration forms, which contain compounds of the general formula (I)

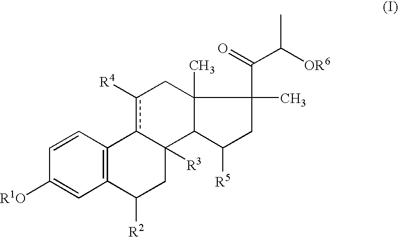

and methods for contraception by administering these administration forms. The invention additionally relates to compounds of the general formula (I) as medication and the use of these compounds for the production of drugs for hormone replacement therapy.

The invention relates to pharmaceutical compositions and administration forms, which contain compounds of the general formula (I)

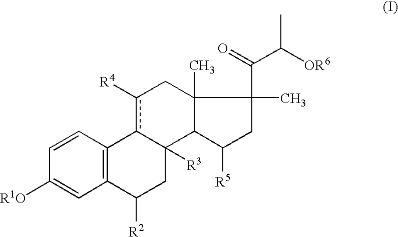

and methods for contraception by administering these administration forms. The invention additionally relates to compounds of the general formula (I) as medication and the use of these compounds for the production of drugs for hormone replacement therapy.

One of the most frequently used methods for contraception is the administration or application of steroid hormones such as oestrogens in combination with progestational agents. However, many of the currently used commercially available contraceptives frequently have side-effects, which can mean intake of the contraceptive has to be interrupted, and therefore no further effective therapy or contraception is assured.

The object of the invention is to make compounds available that are suitable as pharmaceutical active substances and have advantages over conventional pharmaceutical active substances. The pharmaceutical active substances should be suitable in particular for contraception and/or hormone replacement therapy.

This object is achieved by the subject of the patent claims.

It has been surprisingly found that the compounds of the general formula (I) have an affinity to the human progesterone receptor and are therefore suitable in particular as pharmaceutical active substances, e.g. for hormone replacement therapy or for contraception.

The invention relates to a pharmaceutical composition containing
at least one physiologically compatible adjuvant and
at least one compound of the general formula (I)

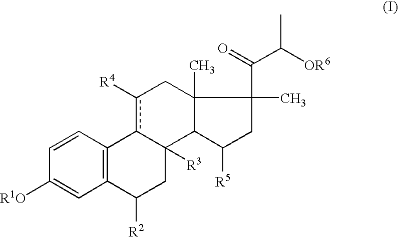

wherein
R¹ and R⁶, respectively independently of one another, stand for —H, —C₁-C₁₂ hydrocarbon or —CO—C₁-C₁₂ hydrocarbon;
R², R³, R⁴ and R⁵, respectively independently of one another, stand for —H, —OH, —O—C₁-C₁₂ hydrocarbon or —O—CO—C₁-C₁₂ hydrocarbon; [symbol]
stands for a single bond or a double bond;
wherein the —C₁-C₁₂ hydrocarbon, respectively independently of one another, can be aliphatic and/or aromatic; and is unsubstituted or is substituted with one or more substituents, which are selected independently of one another from the group comprising halogen and —OH;
in the form of a stereoisomer or mixture thereof, respectively in the form of the free compounds or physiologically compatible salts and/or solvates thereof.

Where reference is made to compounds of the general formula (I) for the purposes of the description, the pharmaceutically compatible salts or solvates are also included, even if this is not expressly mentioned.

In the sense of the description a —C₁-C₁₂ hydrocarbon should preferably be understood to be a residue that contains carbon and hydrogen atoms and 1, 2, 3, 4, 5, 6, 7, 8, 9, 10, 11 or 12 carbon atoms.

The term “hydrocarbon” is known to the person skilled in the art and is defined, for example, in G. P. Moss, Glossary of class names of organic compounds and reactive intermediates based on structure, Pure & Applied Chemistry 1995 (67) 1307-1375.

The hydrocarbon is preferably aliphatic or aromatic or contains both an aliphatic and an aromatic component. The hydrocarbon is preferably saturated or mono- or polyunsaturated. If it is an aliphatic hydrocarbon, then this can be acyclic and/or cyclic (alicyclic). If it is an acyclic hydrocarbon, then this can be linear or branched. Aliphatic hydrocarbons, i.e. linear or branched acyclic hydrocarbons and cyclic (alicyclic) hydrocarbons, can be respectively saturated or mono- or polyunsaturated.

Therefore, in the sense of the description an aliphatic hydrocarbon is preferably understood to be an acyclic or cyclic (alicyclic), saturated or mono- or polyunsaturated hydrocarbon residue, which is not aromatic. Moreover, an acyclic aliphatic hydrocarbon can preferably be unbranched (linear) or branched. If the aliphatic hydrocarbon is unsaturated, then it can preferably have at least one double bond and/or at least one triple bond, preferably 1, 2 or 3 double bonds and/or triple bonds. Suitable saturated or unsaturated aliphatic —C₁-C₁₂ hydrocarbon residues are, for example, methyl, ethyl, n-propyl, iso-propyl, n-butyl, iso-butyl, sec-butyl, tert-butyl, n-pentyl, neo-pentyl, n-hexyl, vinyl, allyl, ethinyl, cyclopropyl, cyclobutyl, cyclopentyl, cyclohexyl, cycloheptyl, cyclopentenyl and cyclohexenyl.

An unsaturated hydrocarbon can have one or more conjugated or non-conjugated C═C double or C≡C triple bonds (e.g. —CH═CH—CH═CH₂, —C≡C—C≡CH), or at the same time have one or more C═C double bonds as well as one or more C≡C triple bonds (e.g. —CH═CH—CH₂—C≡CH), which can again be conjugated or non-conjugated.

The terms “aliphatic” and “alicyclic” are known to the person skilled in the art and are defined, for example, in G. P. Moss, Glossary of class names of organic compounds and reactive intermediates based on structure, Pure & Applied Chemistry 1995 (67) 1307-1375. Saturated, linear or branched, acyclic aliphatic hydrocarbons are usually also referred to as “alkyl” (e.g. methyl, ethyl, propyl, butyl). Accordingly, unsaturated, linear or branched, acyclic aliphatic hydrocarbons that have at least one C═C double bond are usually also referred to as “alkenyl” (e.g. ethenyl, propenyl, vinyl, allyl) and unsaturated, linear or branched, acyclic aliphatic hydrocarbons that have at least one C≡C triple bond are usually also referred to as “alkinyl” (e.g. ethinyl).

Saturated, cyclic aliphatic hydrocarbons are usually also referred to as “cycloalkyl” (e.g. cyclopropyl, cyclobutyl, cyclopentyl, cyclohexyl). Unsaturated, cyclic aliphatic hydrocarbons that have at least one C═C double bond are usually also referred to as “cycloalkenyl” (e.g. cyclopropenyl, cyclohexenyl).

The terms “alkyl”, “alkenyl”, “alkinyl”, “cycloalkyl” and “cycloalkenyl” are known to the person skilled in the art and are defined, for example, in G. P. Moss, Glossary of class names of organic compounds and reactive intermediates based on structure, Pure & Applied Chemistry 1995 (67) 1307-1375.

In the sense of the description the aliphatic hydrocarbon can preferably be formed from both an alicyclic and an acyclic component, which can in turn be linear or branched. In this context, the residues cyclopentylmethyl, cyclohexylethyl, methylcyclopentyl and ethylcyclohexyl are mentioned as examples.

Aromatic hydrocarbons are known to the person skilled in the art. The aromatic hydrocarbon can be uncondensed (not anellated) or condensed (anellated). The terms “aromatic” and “anellated” are known to the person skilled in the art and are defined, for example, in P. Muller, Glossary of terms used in physical organic chemistry, Pure & Applied Chemistry 1994 (66) 1077-1184. A suitable aromatic hydrocarbon that is uncondensed (not anellated) is phenyl, for example. Naphthyl is an example of a condensed (anellated) aromatic hydrocarbon.

In the sense of the description, an aliphatic and aromatic hydrocarbon is preferably understood to be a residue, which contains both an aliphatic component and an aromatic component, wherein the terms aliphatic and aromatic hydrocarbon are defined as described above. Suitable residues, which contain both an aliphatic and an aromatic hydrocarbon, are, for example, benzyl, methylphenyl, dimethylphenyl, mesityl, phenethyl, ethylphenyl, phenylpropyl, propylphenyl, naphthylmethyl, methylnaphthyl, naphthylethyl and ethyl-naphthyl.

In the sense of the description the hydrocarbon residue of the functional group —CO—C₁-C₁₂-hydrocarbon is preferably defined as described above. The —CO—C₁-C₁₂ hydrocarbon residue is preferably selected from the group comprising acetyl, propionyl, butyryl, isobutyryl, pentanoyl, 2-methylbutanoyl, 3-methylbutanoyl, pivaloyl, hexanoyl, heptanoyl, acryloyl, but-3-enoyl, benzoyl, 2-phenylacetyl and naphthoyl.

In the sense of the description the hydrocarbon residue of the functional group —O—C₁-C₁₂-hydrocarbon is preferably defined as described above. The —O—C₁-C₁₂ hydrocarbon residue is preferably selected from the group comprising methoxy, ethoxy, propoxy, iso-propoxy, n-butoxy, iso-butoxy, sec-butoxy, tert-butoxy, n-pentyloxy, neo-pentyloxy, n-hexyloxy, n-heptyl, vinyloxy, allyloxy, phenoxy, benzyloxy, phenethoxy, naphthalen-1-yloxy and naphthalen-2-yloxy.

In the sense of the description the hydrocarbon residue of the functional group —O—CO—C₁-C₁₂-hydrocarbon is preferably defined as described above. The —O—CO—C₁-C₁₂ hydrocarbon residue is preferably selected from the group comprising acetoxy, propionyloxy, butyryloxy, isobutyryloxy, pentanoyloxy, 2-methylbutanoyloxy, 3-methylbutanoyloxy, pivaloyloxy, hexanoyloxy, heptanoyloxy, acryloyloxy, but-3-enoyloxy, benzoyloxy, 2-phenylacetoxy and naphthoyloxy.

In the sense of the description, the term “halogen” is preferably understood to be a residue selected from the group comprising —F, —Cl, —Br and —I.

In the sense of the description, the term “stereoisomer” preferably covers enantiomers and diastereomers. In the sense of the description the term “epimer” is preferably covered by the term “diastereomer”.

In the sense of the description the term “mixture of stereoisomers” preferably stands for the racemate or mixtures of enantiomers and/or diastereomers in any desired ratio.

The terms “enantiomer”, diastereomer”, “epimer” and “racemate” are known to the person skilled in the art and are defined, for example, in G. P. Moss, Basic terminology of stereochemistry, Pure & Applied Chemistry 1996 (68) 2193-2222.

In the sense of the description the term “physiologically compatible salt” is preferably understood to be salts of the compounds according to the invention, which are deemed toxicologically safe—in particular when applied to humans and/or mammals.

In a preferred embodiment the pharmaceutical composition according to the invention contains at least one physiologically compatible adjuvant and at least one compound of the general formula (II)

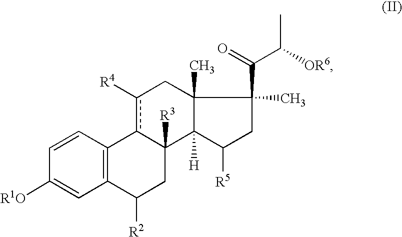

wherein
R¹, R², R³, R⁴, R⁵, R⁶ and [symbol] respectively have the abovementioned meaning; in the form of the free compounds or physiologically compatible salts and/or solvates thereof.

In a preferred embodiment, in the compounds of the general formula (I) or (II) R², R³, R⁴ and R⁵, respectively independently of one another, are selected from the group comprising —H, —OH, methoxy, ethoxy, propoxy, iso-propoxy, n-butoxy, iso-butoxy, sec-butoxy, tert-butoxy, phenoxy, benzyloxy, acetoxy, propionyloxy, butyryloxy, isobutyryloxy, pivaloyloxy, hexanoyloxy, heptanoyloxy and benzoyloxy; more preferred —H or —OH, particularly preferred —H.

In another preferred embodiment, in the compounds of the general formula (I) or (II) R¹ and R⁶, respectively independently of one another, are selected from the group comprising —H, methyl, ethyl, n-propyl, iso-propyl, n-butyl, iso-butyl, sec-butyl, tert-butyl, phenyl, benzyl, acetyl, propionyl, butyryl, isobutyryl, pentanoyl, pivaloyl, hexanoyl, heptanoyl and benzoyl; more preferred —H, methyl, ethyl, n-propyl, iso-propyl, n-butyl, tert-butyl, acetyl, n-propionyl, n-butyryl, pivaloyl, n-hexanoyl, n-heptanoyl and benzoyl; more preferred —H or acetyl; particularly preferred —H.

In a particularly preferred embodiment, in the compounds of the general formula (I) or (II) R², R³, R⁴ and R⁵, respectively independently of one another, are —H or —OH, particularly preferred —H; and R¹ and R⁶ are respectively —H or acetyl, particularly preferred —H, wherein “ [symbol] ” is preferably a single or double bond, particularly preferred a double bond.

In a further preferred embodiment, the pharmaceutical composition according to the invention contains a compound of the general formula (III)

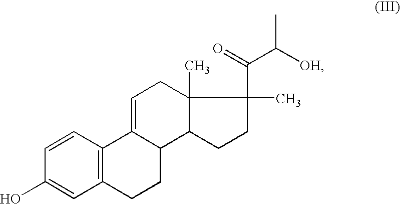

in the form of a stereoisomer or mixture thereof, i.e. 2-hydroxy-1-(3-hydroxy-13,17-dimethyl-7,8,12,13,14,15,16,17-octahydro-6H-cyclopenta[α]phenanthren-17-yl)propan-1-one, respectively in the form of the free compounds or physiologically compatible salts and/or solvates thereof.

In addition, it is particularly preferred if the pharmaceutical composition according to the invention contains the compound of the general formula (IV)

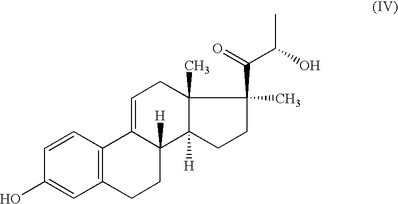

i.e. (S)-2-hydroxy-1-((8S,13S,14S,17S)-3-hydroxy-13,17-dimethyl-7,8,12,13,14,15,16,17-octahydro-6H-cyclopenta[a]phenanthren-17-yl)propan-1-one or (2S,17S)-17-(2-hydroxypropanoyl)-3-hydroxy-17-methylestra-1,3,5(10),9(11)-tetraene, in the form of the free compound, physiologically compatible salts and/or solvates thereof.

If the pharmaceutical composition according to the invention contains a mixture of two enantiomers of the general formula (I), (II), (III) or (IV), the enantiomer excess (ee) amounts to preferably at least 25% ee, more preferred at least 50% ee, further preferred at least 75% ee, most preferred at least 90% ee, and in particular at least 98% ee or 100% ee.

If the pharmaceutical composition according to the invention contains two or more diastereomers of the general formula (I), (II), (III) or (IV), the diastereomer excess (de) amounts to preferably at least 25% de, more preferred at least 50% de, further preferred at least 75% de, most preferred at least 90% de, and in particular at least 98% de or 100% de.

The terms “enantiomer excess” and “diastereomer excess” are known to the person skilled in the art and are defined, for example, in G. P. Moss, Basic terminology of stereochemistry, Pure & Applied Chemistry 1996 (68) 2193-2222.

Compared to the progesterone reference standard, the compounds of the general formula (I), (II), (III) or (IV) preferably exhibit a relative binding affinity to the recombinant human progestin receptor of at least 8.0, more preferred at least 14, further preferred at least 20, most preferred at least 26 and in particular at least 32. In a particularly preferred embodiment, the relative binding affinity to the progestin receptor amounts to 35±4.0.

In a further preferred embodiment, compared to the dexamethasone reference standard, the compounds of the general formula (I), (II), (III) or (IV) exhibit a relative binding affinity to the recombinant human glucocorticoid receptor of at most 10, more preferred at most 7.0, further preferred at most 5.0, most preferred at most 3.0 and in particular at most 1.0. In a particularly preferred embodiment, the relative binding affinity to the glucocorticoid receptor amounts to 0.70±0.10.

Moreover, compared to the testosterone reference standard, the compounds of the general formula (I), (II), (III) or (IV) exhibit a relative binding affinity to the recombinant human androgen receptor of at most 1.0, more preferred at most 0.50, further preferred at most 0.10, most preferred at most 0.070 and in particular at most 0.050. In a particularly preferred embodiment, the relative binding affinity to the androgen receptor amounts to at most 0.030±0.0050.

Compared to the aldosterone reference standard, the compounds of the general formula (I), (II), (III) or (IV) preferably exhibit a relative binding affinity to the recombinant human mineral corticoid receptor of at most 20, more preferred at most 15, further preferred at most 10, most preferred at most 7.0 and in particular at most 4.0. In a particularly preferred embodiment, the relative binding affinity to the mineral corticoid receptor amounts to 2.0±0.20.

In a further preferred embodiment, compared to the oestradiol reference standard, the compounds of the general formula (I), (II), (III) or (IV) exhibit a relative binding affinity to the recombinant human oestrogen receptor of at most 5.0, more preferred at most 1.0, further preferred at most 0.75, most preferred at most 0.50 and in particular at most 0.25. In a particularly preferred embodiment, the relative binding affinity to the oestrogen receptor amounts to 0.18±0.010.

Methods of determining affinity to the aforementioned receptors are known to the person skilled in the art. In this context, reference can be made, for example, to EP-A 808 845; Lacroix et al., Bioorganic & Medicinal Chemistry 1999 (7) 2329-2341; and D. Philibert et al., Gynecol Endocrinol 1999, 13, 316-26.

The selectivity of the compounds of the general formula (I), (II), (III) or (IV) for the progestin receptor compared to the glucocorticoid receptor preferably amounts to at least 10, more preferred at least 20, further preferred at least 30, most preferred at least 40 and in particular at least 50. The selectivity is calculated from the quotient from the relative binding affinity to the progestin receptor and relative binding affinity to the glucocorticoid receptor.

In a preferred embodiment, the selectivity of the compounds of the general formula (I), (II), (III) or (IV) for the progestin receptor compared to the androgen receptor preferably amounts to at least 100, more preferred at least 500, further preferred at least 700, most preferred at least 900 and in particular at least 1100. The selectivity is calculated from the quotient from the relative binding affinity to the progestin receptor and relative binding affinity to the androgen receptor.

In a further preferred embodiment, the selectivity of the compounds of the general formula (I), (II), (III) or (IV) for the progestin receptor compared to the mineral corticoid receptor preferably amounts to at least 5.0, more preferred at least 8.0, further preferred at least 11, most preferred at least 14 and in particular at least 17. The selectivity is calculated from the quotient from the relative binding affinity to the progestin receptor and relative binding affinity to the mineral corticoid receptor.

The selectivity of the compounds of the general formula (I), (II), (III) or (IV) for the progestin receptor compared to the oestrogen receptor preferably amounts to at least 40, more preferred at least 80, further preferred at least 120, most preferred at least 160 and in particular at least 190. The selectivity is calculated from the quotient from the relative binding affinity to the progestin receptor and relative binding affinity to the oestrogen receptor.

It has been surprisingly found that the compounds of the general formulae (I), (II), (III) and (IV), in particular compounds of the general formulae (III) and (IV), exhibit a high selectivity for the progestin receptor, i.e. they bind to the progestin receptor with high affinity, but bind to other steroid receptors, in particular oestrogen receptors, with low affinity. This is surprising, since a high selectivity for the oestrogen receptor would be expected because of the similarity of the structure of the compounds according to the invention to oestrogen (aromatic ring A as in the three natural oestrogens: oestradiol, oestriol and oestrone), however, this is not the case. Therefore, structurally, the compounds according to the invention are to some extent oestrogen derivatives, which, however, functionally have a progestational effect.

The amount of the at least one compound of the general formula (I), (II), (III) or (IV) preferably amounts to at least 100 μg, more preferred at least 200 μg, further preferred at least 300 μg, most preferred at least 400 μg and in particular at least 500 μg, calculated on the basis of the total weight of the pharmaceutical composition according to the invention. In a preferred embodiment, the amount of the at least one compound of the general formula (I), (II), (III) or (IV) in the pharmaceutical composition according to the invention is in the range of 500 μg to 3000 μg, more preferred 510 μg to 2500 μg, further preferred 525 μg to 2000 μg, most preferred 550 μg to 1500 μg and in particular 600 μg to 900 μg.

In another preferred embodiment, the amount of the at least one compound of the general formula (I), (II), (III) or (IV) in the pharmaceutical composition according to the invention corresponds to an equivalent dose of trimegestone of at least 100 μg, more preferred at least 200 μg, further preferred at least 300 μg, most preferred at least 400 μg and in particular at least 500 μg. In a preferred embodiment, the amount of the at least one compound of the general formula (I), (II), (III) or (IV) in the pharmaceutical composition according to the invention corresponds to an equivalent dose of trimegestone in the range of 500 μg to 3000 μg, more preferred 510 μg to 2500 μg, further preferred 525 μg to 2000 μg, most preferred 550 μg to 1500 μg and in particular 600 μg to 900 μg.

In this case, the equivalent dose of the at least one compound of the general formula (I), (II), (III) or (IV) compared to trimegestone is selected so that the progestational activity corresponds to that which the administration of trimegestone would cause in the specified amount. Suitable methods for determining the equivalent dose are known to the person skilled in the art.

The pharmaceutical composition preferably contains the at least one compound of the general formula (I), (II), (III) or (IV) in an amount of 0.0010 to 99.999% by wt., more preferred 0.10 to 99.9% by wt., further preferred 0.50 top 75% by wt., most preferred 1.0 to 50% by wt., and in particular 2.0 to 25% by wt., respectively calculated on the basis of the total weight of the pharmaceutical composition.

Besides at least one compound of the general formula (I), (II), (III) or (IV), possibly respectively in the form of a stereoisomer or mixture thereof, respectively in the form of the free compounds or physiologically compatible salts and/or solvates thereof, the pharmaceutical composition according to the invention additionally contains one or more adjuvants, which are preferably selected from the group comprising salt forming agents, buffers, emulsifiers, embedding media, thickening agents, penetration promoters, film-forming agents, binders, glidants, surfactants, softeners, disintegration accelerators, solvents, wetting agents, gelling agents, preservatives, stabilisers (reducing agents, antioxidants), mould release agents, fillers, lubricants, chelating agents, flavouring additives, fragrances and colouring agents.

Suitable buffers that can be used for the pharmaceutical composition are known to the person skilled in the art. Thus, a phosphate, acetate or carbonate buffer can be used as buffer, for example.

Emulsifiers are preferably added in such amounts that they allow a uniform mixture of the components of the pharmaceutical composition according to the invention. Usual emulsifiers preferably comprise anionic, cationic and/or non-ionic surfactants. Examples of such emulsifiers preferably comprise sodium stearate, sodium lauryl-5-sulphate, sodium acetyl sulphate, polyoxyethylene stearate, polyoxyethylene sorbitane monostearate, sorbitane, propylene glycol monostearate and/or ethoxylated lanolin. The emulsifier is preferably used in an amount of 0.1 to 10% by wt., calculated on the basis of the total weight of the pharmaceutical composition according to the invention.

Examples of embedding media are carnauba wax, montan glycol wax, stearin palmitic acid, glycerol trioleate and cetyl stearyl alcohol.

Thickening agents that can preferably be contained in the composition according to the invention comprise, for example, candelilla, carnauba and microcrystalline waxes, carbomer and polyethylene thickeners. The thickening agent is preferably used in an amount of 0.50 to 2.0% by wt., calculated on the basis of the total weight of the pharmaceutical composition according to the invention.

Suitable penetration promoters in the sense of the description preferably comprise penetration promoters selected from the group comprising acid amides and amines. The use of urea as penetration promoter is particularly preferred. The penetration promoter is preferably used in an amount of 0.50 to 10% by wt., calculated on the basis of the total weight of the pharmaceutical composition according to the invention.

Examples of film-forming agents are shellac, methylcellulose, hydroxypropyl methylcellulose (HPMC), hydroxypropyl cellulose, hydroxy ethyl cellulose, ethyl cellulose, polyacrylates and polymethacrylates.

Suitable binders are, for example, hydroxypropyl cellulose, starch, cellulose ether, polyvinyl pyrrolidone (povidone), hydroxypropyl methylcellulose, gelatin and sugars (e.g. saccharose, glucose syrup). The binders can represent a proportion by weight of preferably 0.50 to 5.0% by wt. calculated on the basis of the total weight of the pharmaceutical composition according to the invention.

Suitable glidants are, for example, talc, long-chain fatty acids such as stearic acid and palmitic acid, salts thereof such as magnesium stearate and calcium stearate, polyethylene glycol and hydrogenated vegetable oils. The glidants can represent a proportion by weight of preferably 0.25 to 3.0% by wt. calculated on the basis of the total weight of the pharmaceutical composition according to the invention.

A suitable flow improver is e.g. colloidal silicon dioxide. The flow improvers can represent a proportion by weight of preferably 0.10 to 3.0% by wt. calculated on the basis of the total weight of the pharmaceutical composition according to the invention.

In addition, surface-active substances (surfactants) that have non-ionic, cationic, anionic and/or ampholytic properties can also be included. The preferred non-ionic surface-active substances used for the pharmaceutical composition preferably comprise sorbitane esters, polyoxyethylene sorbitane esters, glycerol esters, polyoxyethylene glycerol ethers, polyglycerol fatty acid esters, glycerol fatty acid esters, polyoxyethylene glycerol fatty acid esters, polyoxyethylene-branched alkyl ethers, polyoxyethylene alkyl ethers, polyoxyethylene-hydrogenated castor oil fatty acid esters, and/or polyoxyethylene alkylaryl ethers.

Anionic surface-active substances preferably comprise salts, such as diethanolamine salt and trietholanamine salt, higher fatty acids, ether carboxylic acid alkaline salts and N-acyl amino acid salts. The surface-active substance is preferably used in an amount of 0.10 to 10% by wt. calculated on the basis of the total weight of the pharmaceutical composition according to the invention.

Softeners can also be included in the pharmaceutical composition according to the invention. Usual softeners are preferably selected from the group comprising oils and waxes, silicon oils, triglyceride esters, acetoglyceride esters, ethoxylated glycerides, alkyl esters, alkenyl esters, fatty acids, fatty alcohols, fatty alcohol esters, lanolin and derivatives thereof, polyhydrogenated alcohols and ethers thereof, polyhydrogenated alcohol esters, wax esters, beeswax derivatives, vegetable waxes, phospholipids, sterols and amides. The softeners are preferably used in an amount of 1.0 to 25% by wt., calculated on the total weight of the pharmaceutical composition according to the invention.

Disintegration accelerators (tablet disintegrants) are added to a pharmaceutical composition to promote the disintegration of an administration form produced from the composition. Suitable disintegrants are, for example, modified or unmodified starches, clay minerals, cross-linked polyvinyl pyrrolidone, modified or unmodified cellulose, gums or algins. The tablet disintegrants can represent a proportion by weight of preferably 5.0 to 50% by wt., more preferred 5.0 to 15% by wt., calculated on the basis of the total weight of the pharmaceutical composition according to the invention.

Solvents such as water, ethanol, mixtures of water and ethanol, propylene glycol or glycerol, can be included in the pharmaceutical composition according to the invention. However, it is preferred if the pharmaceutical composition is free from solvents, i.e. it has a (residual) moisture of less than 10% by wt., more preferred less than 5.0% by wt., further preferred less than 2.0% by wt., most preferred less than 1.0% by wt. and in particular less than 0.50% by wt., calculated on the basis of the total weight of the pharmaceutical composition according to the invention.

An example of a wetting agent is glycerine.

Suitable gelling agents for the compositions according to the invention preferably comprise natural or synthetic polymers. Natural polymers are preferably selected from the group comprising agar agar, alginic acid, alginate, carbomer, carrageenan, dextrins, gelatins, guar gum, gum arabic, keratin, pectin, shellac, (possibly modified) starch, traganth gum, xanthan gum and derivatives thereof. Preferred synthetic polymers that can be used as gelling agent for the composition according to the invention are selected from the group comprising acrylic acid polymers, carbomers, polyacrylamides and alkylene oxide polymers. The gelling agents are preferably used in an amount of 0.10 to 5.0% by wt., calculated on the basis of the total weight of the pharmaceutical composition according to the invention.

Preservatives can also be included in the pharmaceutical composition according to the invention. Examples of preservatives are alcohols (e.g. ethanol, phenylethyl alcohol, benzyl alcohol), acids (e.g. sorbic acid or benzoic acid) and phenol derivatives (e.g. phenol, chlorocresol). The preservatives are preferably used in an amount of 0.0010 to 15% by wt., calculated on the basis of the total weight of the pharmaceutical composition according to the invention.

Antioxidants and/or reducing agents can be used as stabilisers. Suitable reducing agents are, for example, sulphites, mercaptocarboxylic acids, mercaptoamines, mercaptoamides, hydroxides, alcohols, dithio compounds, lithium chloride, 2-mercapto-ethanesulphonic acid, dithionite, formic acid and oxalic acid. The reducing agents are preferably used in an amount of 0.0010 to 2.0% by wt., calculated on the basis of the total weight of the pharmaceutical composition according to the invention.

Suitable antioxidants are, for example, ascorbic acid, butylated hydroxyanisole, butylated hydroxytoluol, calcium-disodium EDTA, lecithin, lactic acid, tocopherol and citric acid. The antioxidant is preferably used in an amount of 0.0010 to 2.0% by wt., calculated on the basis of the total weight of the pharmaceutical composition according to the invention.

Suitable fillers are, for example, lactose, mannite, sorbite, cellulose, microcrystalline cellulose, xylite, dextrose, fructose, starch and sucrose and mixtures thereof. The fillers can represent a proportion by weight of preferably 70 to 95% by wt., calculated on the basis of the total weight of the pharmaceutical composition according to the invention.

Examples of lubricants are stearic acid, magnesium stearate, calcium stearate and zinc stearate.

Examples of chelating agents are citric acid, phenylalanine, sodium calcium edetate and disodium edetate (EDTA-Na₂).

The pharmaceutical composition can also contain fragrances or flavourings. Suitable fragrances or flavourings are, for example, citronellal, citronellol, eucalyptol, geraniol, limonene, myrcenol, pinene, cedrene, eugenol and vanillin. The fragrance or flavouring is preferably used in an amount of 0.0010 to 2.0% by wt., calculated on the basis of the total weight of the pharmaceutical composition according to the invention.

Colouring agents preferably used for the pharmaceutical composition according to the invention comprise:
i) inorganic and organic pigments such as, for example, titanium oxide, zircon oxide, cerium oxide, zinc oxide, iron oxide, Prussian blue, carbon blacks, calcium lakes and aluminium lakes;
ii) fat-soluble colouring agents such as, for example, Sudan red, DC Red 17, DC Green 6, beta-carotene, soy oil, Sudan brown, DC Yellow 11, DC Violet 2, DC Orange 5 and Quinoline Yellow;
iii) water-soluble colouring agents such as, for example, iron sulphates, methylene blue and natural dyes.

The colouring agents or colouring agent mixtures are preferably used in an amount of 0.0010 to 2.0% by wt., calculated on the basis of the total weight of the pharmaceutical composition according to the invention.

In a preferred embodiment, in addition to at least one compound of the general formula (I), (II), (III) or (IV) the pharmaceutical composition according to the invention contains the following adjuvants in the following preferred amounts (percentages given are calculated on the basis of the total weight of the pharmaceutical composition according to the invention):

| | Preferred | More preferred | In particular |
|---|---|---|---|
| Ingredient | [% by wt.] | [% by wt.] | [% by wt.] |
| HPMC | 1.0 to 7.5 | 2.5 to 5.0 | 3.0 to 5.0 |
| Titanium dioxide | 0.1 to 2.0 | 0.5 to 1.5 | 0.7 to 1.2 |
| Starch | 10 to 60 | 20 to 40 | 25 to 35 |
| Lactose | 25 to 80 | 40 to 70 | 50 to 65 |
| Stearic acid | 0.1 to 2.5 | 0.2 to 1.5 | 0.3 to 1.0 |
| Talc | 0.1 to 5.0 | 0.5 to 2.5 | 0.9 to 1.5 |

The production of the pharmaceutical composition according to the invention is conducted using usual means, devices, methods and processes known from the prior art such as those described, for example, in “Remington's Pharmaceutical Sciences”, publisher A. R. Gennaro, 17th Edition, Mack Publishing Company, Easton, Pa., 1985, in particular in Part 8, Chapter 76 to 93.

In a preferred embodiment, besides at least one physiologically compatible adjuvant and at least one compound of the general formula (I), (II), (III) or (IV) the pharmaceutical composition according to the invention additionally contains at least one progestational agent that is preferably selected from the group comprising: allyloestrenol, chlormadinon, cyproterone, danazol, demegestone, desogestrel, dienogest, drospirenon, dydrogesterone, ethisterone, ethynodiol, gestodene, gestonoron, hydroxyprogesterone, levonorgestrel, lynestrenol, medroxyprogesterone, medrogestone, megestrol, methyloestrenolone, methyl-nortestosterone, nomegestrol, norethisterone, norethynodrel, norgestrel, norgestimate, progesterone, promegestone, tibolone, trimegestone, 1β-hydroxytrimegestone and 6β-hydroxytrimegestone.

Preferred pharmaceutically compatible esters of the above-specified progestational agents are acetates (e.g. chlormadinon acetate, medroxyprogesterone acetate, megestrol acetate, norethisterone acetate), capronates (e.g. hydroxyprogesterone capronate) and enanthates (e.g. norethisterone enanthate).

In a preferred embodiment, the pharmaceutical composition additionally contains at least one oestrogen, which is preferably selected from the group comprising: chlorotrianisene, dienoestrol, diethylstilboestrol, oestradiol (17β-oestradiol), oestriol, oestrone, ethinyloestradiol, oestradiol benzoate, hexoestrol, mestranol, metal oestriol?, methylestrenol, promestriene and conjugated oestrogens or their pharmaceutically compatible esters such as e.g. valerates. Ethinyloestradiol or a combination of ethinyloestradiol and oestradiol (17β-oestradiol) can be used as additional oestrogen component.

In a preferred embodiment, the pharmaceutical composition contains a combination of at least one physiologically compatible adjuvant, at least one compound of the general formula (I), (II), (III) or (IV) and at least one additional progestational agent from those specified above and/or at least one of the above-specified oestrogens. It is particularly preferred if the pharmaceutical composition contains a combination of at least one physiologically compatible adjuvant, at least one compound of the general formula (I), (II), (III) or (IV) and ethinyloestradiol or a combination of at least one physiologically compatible adjuvant, at least one compound of the general formula (I), (II), (III) or (IV) and a combination of ethinyloestradiol and oestradiol (17β-oestradiol).

The pharmaceutical composition can be liquid (e.g. solution, dispersion, suspension, emulsion), in paste form, semisolid or solid (e.g. powder, granulate). It is preferably solid.

The pharmaceutical composition according to the invention preferably contains at least one iron-containing preparation, folic acid and/or folinic acid.

Examples of preparations containing iron are iron(II) preparations such as e.g. iron(II) sulphate, iron(II) carbonate, iron(II) chloride, iron(II) tartrate, iron(II) gluconate, iron(II) aspartate, iron(II) glycine sulphate, iron(II) fumarate, iron(II) ascorbate, iron(II) iodate, iron(II) succinate and ammonium iron(II) sulphate; and iron(III) preparations such as e.g. iron(III) sodium citrate, iron(III) oxide-saccharose complex, sodium fereditate, iron(III) hydroxide, dextriferron, iron(III) citrate, chondroitin sulphate-iron(III) complex, iron(III) acetyltransferrin, iron(III) protein succinylate and potassium-iron(III) phosphate-citrate complex.

Folic acid and its derivative can be present in the pharmaceutical composition according to the invention preferably in free form or as a salt, e.g. as calcium folate.

The invention additionally relates to a pharmaceutical administration form comprising the above-described pharmaceutical composition.

The administration form according to the invention is preferably manufactured for once, twice or three-times daily, particularly preferred for once or twice daily administration. In a particularly preferred embodiment, the administration form is manufactured for oral administration. A particularly preferred administration form is one that is preferably for oral administration and contains the above-described pharmaceutical composition that is made available in the form of daily units.

The pharmaceutical administration form according to the invention can preferably be provided as a liquid, semisolid or solid administration form, e.g. in the form of suspensions, ointments, creams, lotions, gels, emulsions, tablets, capsules, coated tablets, powders, suppositories, plasters, vaginal rings, vaginal applicators, implants, intrauterine pessaries, hormone coils, injections or depot ampoules. The administration form according to the invention is preferably in the form of a tablet, film tablet, coated tablet, capsule, pellet formulation, suppository, transdermal plaster or vaginal ring. Suitable embodiments are well known to the person skilled in the art.

In the case where the pharmaceutical administration form is solid, this can also preferably be provided in multiparticulate form, e.g. in the form of microtablets, microcapsules, micropellets, expanding pellets, granules, extrudates, spheres, beads or pellets, possibly filling capsules or compressed into (film) tablets, wherein dry compaction packs are also possible.

In the case where the pharmaceutical administration form is liquid or in paste form, this can preferably be provided as a liquid, foam, cream, gel, paste, balm, spray, ointment, lotion, douche (conditioner), tonic, tincture, milk, mousse, powder for dissolving, emulsion (oil in water, water in oil), serum, oil, shampoo, suspension such as liposomes or nanosomes, or as a dispersion.

The administration form according to the invention can release the at least one compound of the general formula (I), (II), (III) or (IV) immediately (immediate release) or in a controlled manner (controlled release). If the release is controlled, then it can be delayed, for example, sustained or prolonged, or a pulsed release, repeat action release.

If the administration form according to the invention contains adjuvants, then these correspond to the above-specified adjuvants, which can also be used for the formulation of the pharmaceutical composition according to the invention.

In a preferred embodiment, the administration form according to the invention, preferably in the form of daily units, preferably for oral administration, contains the at least one compound of the general formula (I), (II), (III) or (IV) in an amount of 0.0010 to 99.999% by wt., more preferred 0.10 to 99.9% by wt., further preferred 0.50 to 75% by wt., most preferred 1.0 to 50% by wt. and in particular 2.0 to 25% by wt., respectively calculated on the basis of the total weight of the administration form.

The dose of the at least one compound of the general formula (I), (II), (III) or (IV) contained in the administration form according to the invention preferably amounts to at least 100 μg, more preferred at least 200 μg, further preferred at least 300 μg, most preferred at least 400 μg, and in particular at least 500 μg. In a preferred embodiment, the dose of the at least one compound of the general formula (I), (II), (III) or (IV) contained in the administration form according to the invention preferably lies in the range of 500 μg to 3000 μg, more preferred from 510 μg to 2500 μg, further preferred from 525 μg to 2000 μg, most preferred from 550 μg to 1500 μg, and in particular from 600 μg to 900 μg. In a preferred embodiment, the dose of the at least one compound of the general formula (I), (II), (III) or (IV) is present in such an amount that its dose corresponds to the equivalent dose of trimegestone in the above-defined range.

If the at least one compound of the general formula (I), (II), (III) or (IV) is present in the administration form according to the invention in combination with at least one progestational agent, then the dose of oestrogen in the administration form preferably corresponds to an equivalent dose of 100 μg to 5000 μg, more preferred from 250 μg to 4000 μg, further preferred from 500 μg to 3500 μg, most preferred from 750 μg to 3000 μg, and in particular from 1000 μg to 2500 μg of chlormadinon acetate. If several progestational agents are included, then the total dose thereof preferably corresponds to the above-mentioned equivalent doses.

The equivalent dose to chlormadinon can be provided by an equivalent amount of each suitable progestational agent, wherein the amount is selected so that progestational activity corresponds to that which the administration of chlormadinon acetate would cause in the specified amount. It is also possible that two or more different progestational agents are used in an amount that corresponds overall to the specified equivalent dose. Suitable methods for determining the equivalent dose are known to the person skilled in the art.

If the administration form according to the invention additionally contains a progestational agent, the relative quantity ratio of the trimegestone equivalent dose of the at least one compound of the general formula (I), (II), (III) or (IV) to the chlormadinon acetate equivalent dose of the additional progestational agent preferably lies in the range of 30:1.0 to 1.0:50, more preferred in the range of 20:1.0 to 1.0:40, further preferred in the range of 10:1.0 to 1.0:30, most preferred in the range of 5.0:1.0 to 1.0:20 and in particular in the range of 2.0:1.0 to 1.0:10.

If the at least one compound of the general formula (I), (II), (III) or (IV) is present in combination with at least one oestrogen, preferably ethinyloestradiol, then the dose of oestrogen in the administration form preferably corresponds to an equivalent dose of 5.0 μg to 55 μg, more preferred from 10 μg to 50 μg, further preferred from 15 μg to 48 μg, most preferred from 20 μg to 45 μg, and in particular from 22 μg to 40 μg of ethinyloestradiol. If several oestrogens are included, then the total dose thereof preferably corresponds to the abovementioned equivalent doses.

The equivalent dose to ethinyloestradiol can be provided by an equivalent amount of each suitable oestrogen, wherein the amount is selected so that oestrogen activity corresponds to that which the administration of ethinyloestradiol would cause in the specified amount. It is also possible that two or more different oestrogens, e.g. ethinyloestradiol in combination with oestradiol, are used in an amount that corresponds overall to the specified equivalent dose. Suitable methods for determining the equivalent dose are known to the person skilled in the art.

If the administration form according to the invention additionally contains an oestrogen, the relative quantity ratio of the trimegestone equivalent dose of the at least one compound of the general formula (I), (II), (III) or (IV) to the ethinyloestradiol equivalent dose of the additional oestrogen preferably lies in the range of 60:1.0 to 1.5:1.0, more preferred in the range of 40:1.0 to 1.4:1.0, further preferred in the range of 20:1.0 to 1.3:1.0, most preferred in the range of 10.0:1.0 to 1.3:1.0 and in particular in the range of 5.0:1.0 to 1.2:1.0.

Particularly preferred embodiments for combinations of a daily dose X of at least one compound of the general formula (I), (II), (III) or (IV) with the daily doses Y of ethinyloestradiol, which can be contained in the pharmaceutical administration form according to the invention, are summarised in the following table:

| Compound of general | |
|---|---|
| formula (I), (II), (III) or (IV) | Ethinyloestradiol |
| 500 ≦ X ≦ 3.000 μg | 10 μg ≦ Y ≦ 50 μg |
| 510 ≦ X ≦ 2.500 μg | 12 μg ≦ Y ≦ 48 μg |
| 525 ≦ X ≦ 2.000 μg | 15 μg ≦ Y ≦ 45 μg |
| 550 ≦ X ≦ 1.500 μg | 18 μg ≦ Y ≦ 42 μg |
| 600 ≦ X ≦ 900 μg | 20 μg ≦ Y ≦ 40 μg |

In a preferred embodiment, the at least one compound of the general formula (I), (II), (III) or (IV) is present in such an amount that its dose corresponds to the equivalent dose X of trimegestone in the above-defined range.

Particularly preferred embodiments for combinations of the daily dose X of at least one compound of the general formula (I), (II), (III) or (IV) with the daily doses Y of ethinyloestradiol and the daily dose Z of oestradiol (17β-oestradiol), which can be contained in the pharmaceutical administration form according to the invention, are summarised in the following table:

| Compound of | | |
|---|---|---|
| general formula | | |
| (I), (II), (III) or (IV) | Ethinyloestradiol | Oestradiol |
| 500 ≦ X ≦ 3000 μg | 1.0 μg ≦ Y ≦ 10 μg | 1000 μg ≦ Z ≦ 10000 μg |
| 510 ≦ X ≦ 2500 μg | 2.0 μg ≦ Y ≦ 10 μg | 1100 μg ≦ Z ≦ 9000 μg |
| 525 ≦ X ≦ 2000 μg | 3.0 μg ≦ Y ≦ 9.5 μg | 1200 μg ≦ Z ≦ 8000 μg |
| 550 ≦ X ≦ 1500 μg | 4.0 μg ≦ Y ≦ 9.5 μg | 1300 μg ≦ Z ≦ 7000 μg |
| 600 ≦ X ≦ 900 μg | 5.0 μg ≦ Y ≦ 9.0 μg | 1400 μg ≦ Z ≦ 6000 μg |

In a preferred embodiment, the at least one compound of the general formula (I), (II), (III) or (IV) is present in such an amount that its dose corresponds to the equivalent dose X of trimegestone in the above-defined range.

The invention additionally relates to a cosmetic composition, which contains at least one compound of the general formula (I), (II), (III) or (IV). The cosmetic composition is preferably for skin and/or hair care, preferably by topical application.

Suitable cosmetic adjuvants are preferably the usual adjuvants of such compositions. In this context reference can be made, for example, to the following in its full scope: H. P. Fiedler, Lexikon der Hilfsstoffe für Pharmazie, Kosmetik und angrenzende technische Gebiete [Glossary of adjuvants for pharmacy, cosmetics and associated technical fields], Editio Cantor Aulendorff, 2002. The above-specified adjuvants that can also be included in the pharmaceutical compositions according to the invention can preferably be used. These adjuvants are physiologically compatible and the amounts of the respective components are preferably selected so that the cosmetic composition according to the invention complies with EU cosmetic guideline 76/768/EEC or EU Guideline 95/17/EC.

The selection of adjuvants and also the amounts thereof to be used is dependent on whether the pharmaceutical or cosmetic composition according to the invention is for oral, topical, subcutaneous, parenteral, intradermal, vaginal or local application or administration, wherein an oral, vaginal, subcutaneous or transdermal application is particularly preferred. Delivery forms to be applied orally or percutaneously can also release the compound of the general formula (I) in a delayed manner.

The invention additionally relates to compounds of the general formulae (I) and (II) as defined above, on condition that if R¹, R², R³, R⁴ and R⁵ are respectively —H, R⁶ is not —CO—CH₃ (acetyl) (cf. I. Lacroix et al., Bioorg. Med. Chem. 1999, 7, 2329-41, compound II), as well as the compounds of the general formulae (III) and (IV) as defined above.

The above statements and definitions relating to the compounds of the general formulae (I), (II), (III) and (IV) of the pharmaceutical composition according to the invention also apply analogously to the corresponding compounds according to the invention.

The invention additionally relates to a compound of the general formula (I), (II), (III) or (IV) as defined above as medication.

The invention additionally relates to a compound of the general formula (I), (II), (III) or (IV) as defined above or the use thereof, possibly in combination with at least one oestrogen and/or a further progestational agent, for the treatment and/or prevention of at least one of the complaints or diseases selected from the group comprising rosacea; psoriasis; menstrual disorders; dysmenorrhoea (painful periods); disorders related to the menstrual cycle such as endometriosis, polycystic ovary syndrome (PCOS), uterine myoma, functional cysts, menstrual cycle-related mood swings, premenstrual dysphoric disorder (PMDD), premenstrual syndrome (PMS) and headache/migraine; disorders influenced by the menstrual cycle such as epilepsy, multiple sclerosis, diabetes mellitus, depression, schizophrenia, asthma and Parkinson's disease: and androgen-induced disorders such as seborrhoea, acne, androgenic alopecia and hirsutism.

The invention additionally relates to the compound of the general formula (I), (II), (III) or (IV) according to the invention or the use thereof, possibly in combination with at least one oestrogen and/or at least one progestational agent, for hormone replacement therapy (HRT).

The invention additionally relates to the use of at least one compound of the general formula (I), (II), (III) or (IV) as defined above, possibly in combination with at least one oestrogen and/or a further progestational agent, for the production of a pharmaceutical composition or administration form described above for the treatment and/or prevention of at least one of the complaints or diseases selected from the group comprising rosacea; psoriasis; menstrual disorders; dysmenorrhoea (painful periods); disorders related to the menstrual cycle such as endometriosis, polycystic ovary syndrome (PCOS), uterine myoma, functional cysts, menstrual cycle-related mood swings, premenstrual dysphoric disorder (PMDD), premenstrual syndrome (PMS) and headache/migraine; disorders influenced by the menstrual cycle such as epilepsy, multiple sclerosis, diabetes mellitus, depression, schizophrenia, asthma and Parkinson's disease: and androgen-induced disorders such as seborrhoea, acne, androgenic alopecia and hirsutism.

The invention additionally relates to the use of a compound of the general formula (I), (II), (III) or (IV) according to the invention, possibly in combination with at least one oestrogen and/or at least one progestational agent, for the production of a pharmaceutical composition or an administration form described above for hormone replacement therapy (HRT), wherein the pharmaceutical composition and administration form are defined as described above.

The invention additionally relates to a method for contraception comprising the administration of an administration form according to the invention as defined above to women of child-bearing age on at least 21 consecutive days, beginning on day 1 of the menstrual cycle. The administration form according to the invention is preferably administered orally in this case.

In a preferred embodiment of the method according to the invention, at least one compound of the general formula (I), (II), (III) or (IV) in combination with at least one oestrogen is administered on at least one, preferably on all of the at least 21 consecutive days, wherein the oestrogen is preferably selected from the group comprising: chlorotrianisene, dienoestrol, diethylstilboestrol, oestradiol (17β-oestradiol), oestriol, oestrone, ethinyloestradiol, oestradiol benzoate, hexoestrol, mestranol, metal oestriol?, methylestrenol, promestriene and conjugated oestrogens or their pharmaceutically compatible esters such as e.g. valerates. Ethinyl-oestradiol or a combination of ethinyloestradiol and oestradiol (17 β-oestradiol) can be used in particular as additional oestrogen component. It is particularly preferred if the additional oestrogen component comprises ethinyloestradiol or a combination of ethinyloestradiol and oestradiol (17β-oestradiol), wherein the amount of the oestrogen component preferably corresponds to an equivalent dose of 5.0 μg to 55 μg, more preferred 10 μg to 50 μg, further preferred 15 μg to 48 μg, most preferred 20 μg to 45 μg, and in particular 22 μg to 40 μg of ethinyloestradiol. If several oestrogens are used, then the total daily dose thereof preferably corresponds to the aforementioned equivalent doses.

In a particularly preferred embodiment of the method according to the invention, the oestrogen is not administered without the administration of at least one compound of the general formula (I), (II), (III) or (IV) on any of the at least 21 consecutive days.

In a preferred embodiment of the method according to the invention, the daily dose of the at least one compound of the general formula (I), (II), (III) or (IV) is the same on each of the at least 21, more preferred at least 22, further preferred at least 23, most preferred at least 24, and in particular at least 25 consecutive days (=single−phase schedule), wherein the administration preferably occurs respectively in combination with at least one oestrogen.

In another preferred embodiment of the method according to the invention, the at least 21, more preferred at least 22, further preferred at least 23, most preferred at least 24, and in particular at least 25 consecutive days are divided into two, three or more groups of days, wherein on all days within a group the daily dose of the at least one compound of the general formula (I), (II), (III) or (IV) is the same, but on consecutive days of different groups the daily dose of the at least one compound of the general formula (I), (II), (III) or (IV) is different (=multiphase schedule), and wherein the administration preferably occurs respectively in combination with at least one oestrogen.

Preferred schedules are given in the following table, wherein the daily dose of the at least one compound of the general formula (I), (II), (III) or (IV) is A1, A2 or A3 and the daily dose of the at least one oestrogen is B:

| | | Number of Phases | | | | | | | |
|---|---|---|---|---|---|---|---|---|---|
| | | 1 | 2 | 2 | 3 | 3 | 3 | 4 | 4 |
| | | Embodiment no. | | | | | | | |
| | | 1 | 2₁ | 2₂ | 3₁ | 3₂ | 3₃ | 4₁ | 4₂ |
| | | Total duration [days of 28] | | | | | | | |
| | | 21-25 | 21-25 | 21-25 | 21-25 | 21-25 | 21-25 | 21-25 | 21-25 |
| 1 | Duration [days] | 21-25 | 7-13 | 7-13 | 3-8 | 3-8 | 3-8 | 3-8 | 3-8 |
| | Dose of compound formula | A1 | A2 | A1 | A2 | A2 | A1 | A2 | A2 |
| | (I), (II), (III) or (IV) | | | | | | | | |
| | Oestrogen dose (equivalent | B | B | B | B | B | B | B | B |
| | dose to ethinyloestradiol) | | | | | | | | |
| 2 | Duration [days] | | 12-18 | 12-18 | 4-15 | 4-15 | 4-15 | 4-15 | 4-15 |
| | Dose of compound formula | | A1 | A2 | A2 | A1 | A2 | A1 | A2 |
| | (I), (II), (III) or (IV) | | | | | | | | |
| | Oestrogen dose (equivalent | | B | B | B | B | B | B | B |
| | dose to ethinyloestradiol) | | | | | | | | |
| 3 | Duration [days] | | | | 4-15 | 4-15 | 4-15 | 4-15 | 4-15 |
| | Dose of compound formula | | | | A1 | A2 | A2 | A2 | A1 |
| | (I), (II), (III) or (IV) | | | | | | | | |
| | Oestrogen dose (equivalent | | | | B | B | B | B | B |
| | dose to ethinyloestradiol) | | | | | | | | |
| 4 | Duration [days] | | | | | | | 2-5 | 2-5 |
| | Dose of compound formula | | | | | | | A3 | A3 |
| | (I), (II), (III) or (IV) | | | | | | | | |
| | Oestrogen dose (equivalent | | | | | | | B | B |
| | dose to ethinyloestradiol) | | | | | | | | |

The respective value ranges of the doses for the respective combinations of A1, A2, A3 and B for each individual embodiment of embodiment nos. 1, 2₁, 2₂, 3₁, 3₂, 3₃, 4₁ and 4₂ can be seen from the following table, wherein dosage B of the at least one oestrogen is given as equivalent dose to ethinyloestradiol:

| | Preferred | More preferred | Further preferred | In particular |
|---|---|---|---|---|
| A1 | 510-990 μg | 525-975 μg | 550-950 μg | 550-750 μg |
| A2 | 40-990 μg | 40-750 μg | 120-750 μg | 260-500 μg |
| A3 | 0-990 μg | 0-750 μg | 0-500 μg | 260-500 μg |
| B | 5.0-55 μg | 10-50 μg | 20-45 μg | 25-40 μg |

In a preferred embodiment, the compound of the general formula (I), (II), (III) or (IV) is present in such an amount that the dose thereof corresponds to the equivalent dose A1, A2 or A3 of trimegestone in the above-defined range.

In a preferred embodiment of the method according to the invention the at least one compound of the general formula (I), (II), (III) or (IV) is not administered on all days of the preferably 28-day menstrual cycle. Rather, on those days following the at least 21 consecutive days, it is preferred if
a placebo,

an iron-containing pharmaceutically compatible preparation or
a folic acid-containing preparation is administered;
or no administration occurs.

In this way, the menstrual cycle is ended by the withdrawal bleeding, so that a new menstrual cycle can begin. The menstrual cycle is preferably 28 days.

According to another preferred embodiment of the method according to the invention, however, it is possible that the menstrual cycle is longer than 28 days. This can be achieved according to the invention by discontinuing the at least one compound of the general formula (I), (II), (III) or (IV) (and possibly at least one oestrogen and/or at least one further progestational agent) at a later point in time, so that the withdrawal bleeding also only occurs at a later point in time and therefore the menstrual cycle also only ends at a later point in time. In this embodiment, administration of the at least one compound of the general formula (I), (II), (III) or (IV) preferably occurs on more than 28 consecutive days.

In this embodiment, the (uninterrupted) administration of the at least one compound of the general formula (I), (II), (III) or (IV) preferably occurs on at least 42 or 56, more preferred at least 63, further preferred at least 84, most preferred at least 105 or 112, and in particular at least 126 or 140 consecutive days, so that no triggering of the withdrawal bleeding is intended within this time period. The continuous time period, in which a daily administration of the at least one compound of the general formula (I), (II), (III) or (IV) occurs, can also be even longer according to the invention. Thus, it is fundamentally possible to administer a compound of the general formula (I), (II), (III) or (IV) on all consecutive days over one year or several years without a withdrawal bleeding occurring.

In a preferred embodiment of the method according to the invention, the at least one compound of the general formula (I), (II), (III) or (IV) is administered in combination with at least one further progestational agent on one of the at least 21 consecutive days. The progestational agents used in such a combination as well as their corresponding respective doses correspond to those of the pharmaceutical composition according to the invention specified above.

The method according to the invention is conducted during at least one menstrual cycle. The method according to the invention is preferably conducted during several, in particular during at least 6, consecutive menstrual cycles.

A further aspect of the invention relates to a kit comprising at least one of the above-described administration forms according to the invention. The kit according to the invention is preferably designed respectively for a once daily administration of the administration forms contained therein.

The kit is preferably assembled so that the above-described method for contraception according to the invention can be conducted by means of the administration forms according to the invention that are contained in the kit without having to acquire further administration forms according to the invention that are not contained in the kit. The kit preferably contains a respective administration form for one day, since administration preferably occurs once daily.

If the menstrual cycle is 28 days, then the kit according to the invention preferably comprises as many administration forms as required to administer at least one compound of the general formula (I), (II), (III) or (IV) on at least 21 consecutive days of a 28-day menstrual cycle. If the at least one compound of the general formula (I), (II), (III) or (IV) is administered on less than 28 days, then for the remaining days until the 28 days of the menstrual cycle have elapsed the kit according to the invention can contain either no administration forms at all, iron-containing preparations, folic acid-containing preparations or placebos, preferably an iron-containing preparation. It is necessary in this case that at least one of the administration forms of the kit according to the invention is an administration form according to the invention.

If the menstrual cycle is extended, i.e. if it is more than 28 days, then the number of administration forms contained in the kit according to the invention is increased accordingly, wherein at least one of the administration forms contained therein is again an administration form according to the invention described above.

In a preferred embodiment, the kit according to the invention comprises all administration forms required for the administration of at least one compound of the general formula (I), (II), (III) or (IV) for at least one or two, preferably at least three, more preferred at least four, most preferred at least five, and in particular at least six menstrual cycles.

In a preferred embodiment, the kit according to the invention is designed for a single- or multiphase administration of at least one compound of the general formula (I), (II), (III) or (IV) in combination with an oestrogen. In this case, the menstrual cycle is preferably 28 days. In the case of the two-, three- and four-phase schedules, the daily dose of the at least one compound of the general formula (I), (II), (III) or (IV) and oestrogen is respectively constant on all days within one phase and different on two consecutive days of different phases.

Preferred embodiment nos. 1, 2₁, 2₂, 3₁, 3₂, 3₃, 4₁ and 4₂ of the kit according to the invention comprise 21-25 administration forms according to the invention overall, wherein, depending on the number of phases, these contain the at least one compound of the general formula (I), (II), (III) or (IV) according to the invention in doses A1, A2, A3 and at least one oestrogen in dose B in accordance with the following table:

| | | Number of Phases | | | | | | | |
|---|---|---|---|---|---|---|---|---|---|
| | | 1 | 2 | 2 | 3 | 3 | 3 | 4 | 4 |
| | | Embodiment no. | | | | | | | |
| | | 1 | 2₁ | 2₂ | 3₁ | 3₂ | 3₃ | 4₁ | 4₂ |
| | | Administration forms overall | | | | | | | |
| | | 21-25 | 21-25 | 21-25 | 21-25 | 21-25 | 21-25 | 21-25 | 21-25 |
| 1 | Number of dose units | 21-25 | 7-13 | 7-13 | 3-8 | 3-8 | 3-8 | 3-8 | 3-8 |
| | Dose of compound formula | A1 | A2 | A1 | A2 | A2 | A1 | A2 | A2 |
| | (I), (II), (III) or (IV) | | | | | | | | |
| | Oestrogen dose (equivalent | B | B | B | B | B | B | B | B |
| | dose to ethinyloestradiol) | | | | | | | | |
| 2 | Number of dose units | | 12-18 | 12-18 | 4-15 | 4-15 | 4-15 | 4-15 | 4-15 |
| | Dose or compound formula | | A1 | A2 | A2 | A1 | A2 | A1 | A2 |
| | (I), (II), (III) or (IV) | | | | | | | | |
| | Oestrogen dose (equivalent | | B | B | B | B | B | B | B |
| | dose to ethinyloestradiol) | | | | | | | | |
| 3 | Number of dose units | | | | 4-15 | 4-15 | 4-15 | 4-15 | 4-15 |
| | Dose of compound formula | | | | A1 | A2 | A2 | A2 | A1 |
| | (I), (II), (III) or (IV) | | | | | | | | |
| | Oestrogen dose (equivalent | | | | B | B | B | B | B |
| | dose to ethinyloestradiol) | | | | | | | | |
| 4 | Number of dose units | | | | | | | 2-5 | 2-5 |
| | Dose of compound formula | | | | | | | A3 | A3 |
| | (I), (II), (III) or (IV) | | | | | | | | |
| | Oestrogen dose (equivalent | | | | | | | B | B |
| | dose to ethinyloestradiol) | | | | | | | | |

The respective value ranges of the doses for the respective combinations of A1, A2, A3 and B for each individual embodiment of these embodiment nos. 1, 2₁, 2₂, 3₁, 3₂, 3₃, 4₁ and 4₂ can be seen from the following table, wherein dosage B of the at least one oestrogen is given as equivalent dose to ethinyloestradiol:

| | Preferred | More preferred | Further preferred | In particular |
|---|---|---|---|---|
| A1 | 510-990 μg | 525-975 μg | 550-950 μg | 550-750 μg |
| A2 | 40-990 μg | 40-750 μg | 120-750 μg | 260-500 μg |
| A3 | 0-990 μg | 0-750 μg | 0-500 μg | 260-500 μg |
| B | 5.0-55 μg | 10-50 μg | 20-45 μg | 25-40 μg |

In a preferred embodiment, the at least one compound of the general formula (I), (II), (III) or (IV) is present in such an amount that the dose thereof corresponds to the equivalent dose A1, A2 or A3 of trimegestone in the above-defined range.

The at least one compound of the general formula (I), (II), (III) or (IV) is preferably used in combination with ethinyloestradiol or in combination with ethinyloestradiol and oestradiol (17β-oestradiol).

In the case of the two-, three- and four-phase schedules, the daily dose of the at least one compound of the general formula (I), (II), (III) or (IV) and oestrogen is respectively constant on all days within one phase and different on two consecutive days of different phases.

The following examples serve to explain the invention in more detail, while not interpreting it in a restrictive manner.

### (2S, 17S)-17-(2-hydroxypropanoyl)-3-hydroxy-17-methylestra-1,3,5(10),9(11)-tetraene

The title compound can be obtained by acetylation of 1-β-hydroxytrimegestone, aromatisation of the product thus obtained and then alkaline saponification. For this, the aromatised intermediate stage (cf. I. Lacroix et al., Bioorg. Med. Chem. 1999, 7, 2329-2341, phenolic compound II) can be saponified:

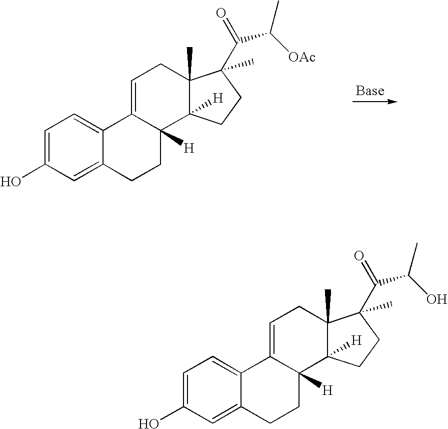

Suitable conditions for alkaline ester saponification are known to the person skilled in the art. In this context, reference can be made, for example, to J. March, Advanced Organic Chemistry, 4th ed., John Wiley & Sons, Inc., 1992, pp 378-383.

### Relative Binding Affinity and Selectivity

The relative binding affinities of (2S,17S)-17-(2-hydroxypropanoyl)-3-hydroxy-17-methyl-estra-1,3,5(10),9(11)-tetraene [compound of the general formula (IV)] to the recombinant human progestin receptor, mineral corticoid receptor, androgen receptor, glucocorticoid receptor and oestrogen receptor were determined experimentally (cf. D. Philibert et al., Gynecol Endocrinol 1999, 13, 316-26).

It was found that (2S,17S)-17-(2-hydroxypropanoyl)-3-hydroxy-17-methylestra-1,3,5(10),9(11)-tetraene has a clear affinity to the progestin receptor with a high selectivity. This is particularly surprising with respect to the oestrogen receptor, since a higher affinity to the oestrogen receptor and thus a lower selectivity for the progestin receptor would be expected in view of the structural similarity between oestrogen and the compounds according to the invention.

1. A pharmaceutical composition comprising at least one physiologically compatible adjuvant and at least one compound of the formula (I) 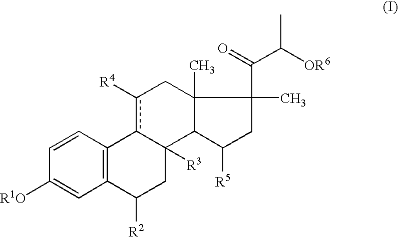
wherein
R¹ and R⁶, respectively independently of one another, are selected from —H, a —C₁-C₁₂ hydrocarbon or a —CO—C₁-C₁₂ hydrocarbon;
R², R³, R⁴ and R⁵, respectively independently of one another, are selected from —H, —OH, a —O—C₁-C₁₂ hydrocarbon or a —O—CO—C₁-C₁₂ hydrocarbon;
wherein the —C₁-C₁₂ hydrocarbon, respectively independently of one another, are aliphatic or aromatic; and is unsubstituted or substituted with one or more substituents, which are selected independently of one another from the group consisting of halogen and —OH;
wherein said compound is optionally in the form of a stereoisomer or mixture thereof, respectively in the form of a free compound or a physiologically compatible salt or solvate thereof.

2. The pharmaceutical composition according to claim 1, wherein R², R³, R⁴ and R⁵ respectively are —H.

3. The pharmaceutical composition according to claim 1, wherein R¹ and R⁶ are —H.

4. The pharmaceutical composition according to claim 1, wherein the compound of the formula (I) has a relative binding affinity to the progestin receptor of at least 8.0 compared to the progesterone reference standard.

5. The pharmaceutical composition according to claim 1, wherein the physiologically compatible adjuvant is selected from the group consisting of salt forming agents, buffers, emulsifiers, embedding media, thickening agents, penetration promoters, film-forming agents, binders, glidants, surfactants, softeners, disintegration accelerators, solvents, wetting agents, gelling agents, preservatives, stabilisers, mould release agents, fillers, lubricants, chelating agents, flavouring additives, fragrances and colouring agents.

6. The pharmaceutical composition according to, additionally comprising oestrogen.

7. The pharmaceutical composition according to claim 6, wherein the oestrogen comprises ethinyloestradiol.

8. A pharmaceutical administration form comprising a pharmaceutical composition according to claim 1.

9. The pharmaceutical administration form according to claim 8, wherein the form is intended for once or twice daily administration.

10. The pharmaceutical administration form according to claim 8, wherein the form is intended for oral administration.

11. The pharmaceutical composition according to claim 1 or, wherein the composition is solid, semisolid or liquid.

12. The compound of formula (I) defined as in claim 1 with the proviso that if R², R³, R⁴ and R⁵ are respectively —H, R⁶ is not —CO—CH₃.

13. The compound of formula (I) as defined in claim 1 wherein the compound is intended as a medication.

14. (canceled)

15. A method for contraception comprising the administration of an administration form according to claim 8 to women of child-bearing age on at least 21 consecutive days beginning on day 1 of the menstrual cycle.
